(19) United States
(12) Patent Application Publication
(10) Pub. No.: US 2005/0013925 A1
(43) Pub. Date: Jan. 20, 2005

(54) Immobilization methods for organic molecules telomers and polymers on solid substrates

(76) Inventors: Melvin Cabey, Midland, MI (US); Yung K. Kim, Midland, MI (US)

Correspondence Address: MCKELLAR STEVENS & HILL PLLC, POSEYVILLE PROFESSIONAL COMPLEX, 784 SOUTH POSEYVILLE ROAD, MIDLAND, MI, 48640, US

(21) Appl. No.: 10/865,708

(22) Filed: Jun. 10, 2004

Related U.S. Application Data
(60) Provisional application No. 60/488,195, filed on Jul. 17, 2003.

(51) Int. Cl. B05D 3/00; C12M 1/34

(52) U.S. Cl. 427/2.11; 435/287.2

(57) ABSTRACT

The immobilization of organic molecules, telomers and polymers on solid substrates. Organic molecules, telomers and polymers are immobilized onto solid substrates, especially particulate materials via covalent bonds that provide greater thermal and organic stability than other methods of immobilization.

This application claims priority from U.S. Provisional application 60/488,195, filed on Jul. 17, 2003.

The invention disclosed and claimed herein deals with the immobilization of organic molecules, telomers and polymers on solid substrates.

Organic molecules, telomers and polymers are immobilized onto solid substrates, especially particulate materials via covalent bonds that provide greater thermal and organic stability than other methods of immobilization.

### BACKGROUND OF THE INNENTION

Immobilization of molecules and/or materials on substrates, such as silicas and polymeric resinous beads are known. However, covalent bonding immobilization on solid substrates requires reactive functional groups on the molecules to be attached, such as —SiOR, —SiX, and the like. While R—OH types of functional groups result in immobilization on solid substrates, the resulting products are not stable because the bond is an R—O—Si—substrate bond which are known to be chemically unstable, for example, are readily hydrolyzable. Such bonds are also subject to other chemical attacks.

Further, immobilization of molecules and higher molecular weight telomers and polymers containing no reactive stable, and covalently bondable groups, have been achieved via adsorption and physical entrapment. These types of immobilized materials lose their activity due to gradual loss of the immobilized molecules via leaching.

Covalently bonded immobilization of non-bondable functional molecules and higher molecular weight telomers and polymers is very difficult if not impossible. Even if higher molecular weight telomers and polymers contain bondable functional groups, covalently bonded immobilization of such telomers and polymers for any meaningful amount are very difficult if not impossible by the known methods.

The prior art methods have a weakness, in that, current covalent immobilization techniques provide very low “actives” loading on the surface of the solid substrates due to limited reactive sites on such solid substrates. For example, covalent immobilization of enzymes gives about 0.02 grams of enzyme/gram of matrix, while adsorption immobilization gives about 1 g of enzyme/gram of matrix (See cf Martin Chaplin, Chapter 3, Methods of Immobilization, Enzyme Technology, London South Bank University.)

### BRIEF DESCRIPTION OF THE DRAWINGS

FIG. 1 is a schematic of a generic reaction to provide the unsaturated groups bound to a silica particle wherein x has a value of 0 to 20; K represents organic molecules, telomers, polymers capable of generating a radical by the use of the radical initiator.

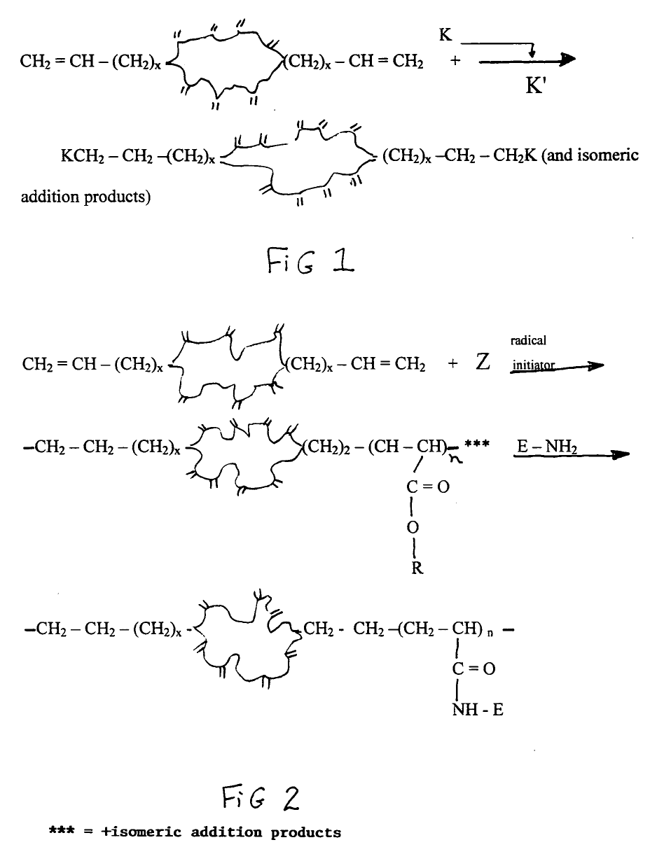

FIG. 2 is a detailed schematic of a generic reaction to provide reactive groups on the material of FIG. 1, wherein 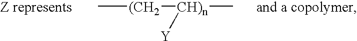
grafted to polyolefins, such as 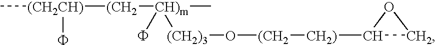
wherein Y represents —C═(O)—O R or reactive functional groups, E is any organic molecule that is capable of reacting with functional groups, such as an epoxy or ester group, and Φ represents a phenyl group.

### THE INVENTION

The present invention deals with the use of free radical chemistry and processes to covalently immobilize non-bondable organic molecules, telomers and polymers on substrates pre-bonded with olefinic groups. The process is simple and efficient and results in covalently immobilized molecules, telomers and polymers on various solid substrates without altering or damaging the organically reactive functional groups such as amino, carboxy, epoxy, and the like, on the organic molecules, telomers and polymers. As a result, the covalently immobilized materials provide a large number of chemically reactive functional groups that can be used to covalently immobilize a large number of molecules, including enzymes, that are useful for many applications.

Thus, what is disclosed and claimed herein is a method of covalently bonding a material selected from the group consisting of organic molecules, telomers, and polymers, on solid substrates, the process comprising contacting the solid substrate with an alkenyl containing silane containing organofunctional moieties and allowing the organofunctional moieties to react with the solid substrate to provide an alkenyl functional group bound to the solid substrate to form a first product.

Thereafter, contacting the first product from just above with a material selected from the group consisting of organic molecules, telomers, and polymers, in the presence of free radicals, to covalently bond the material to the solid substrate.

Another embodiment of this invention is a process in which materials selected from the groups consisting of organic molecules, telomers, and polymers are covalently reacted, in the presence of a free radicals, with alkenyl groups on silanes, wherein the silanes also contain organofunctional moieties, to form a first product, and then reacting the first product of that reaction with a solid substrate using the organofunctional moieties to bind the solid substrate to the first product.

The methods described and claimed herein are novel and utilize olefinic functional groups, in one embodiment, previously bonded onto the solid substrate to bond to C—H groups of organic molecules, telomers or polymers via free radical catalyzed addition reactions to achieve a covalent bonding to the solid substrate. The materials have a broad range of applications from various analytical and other smaller volume applications, to large volume industrial applications as are set forth by way of example, infra.

Organic molecules, telomers and polymers have previously been immobilized onto silica and other solid substrates but reactive groups had to be built onto them in order to bond on the substrate surface.

This invention involves the immobilization of organic molecules, telomers, and polymers via stable, covalent bonding wherein the organic molecules, telomers, and polymers do not normally have reactive groups under ambient conditions. The invention also involves the use of olefinic functional silanes that are attached to solid substrates, such as silica or other silaceous materials via hydrolysis and subsequent reaction with hydroxyl groups on the substrate. Then, the reaction of the olefinic groups of the silanes, with organic molecules, telomers, or polymers, via free radical catalyzed addition of C—H groups on them, is undertaken to bond the organic molecules, telomers, and polymers with the olefinic groups.

Olefinic groups on the silanes include vinyl, allyl, hexenyl, and longer chain length olefinic containing molecules. Such olefinic silanes also include one or more hydrolyzable groups such as, for example, alkoxy groups, such as methoxy, ethoxy, isopropoxy, and the like, halo groups, such as chloro, ester groups such as acetoxy and propoxy, and oximo groups, and the like.

Organic molecules useful in this invention include crown ethers and other aliphatic carbon/hydrogen-containing molecules. Telomers and polymers included in this invention are, for example, polyethylene, polystyrene and almost any other material containing aliphatic C—H groups, and which also include aliphatic C—H groups on aromatic materials, such as toluene. Telomers are defined as the products of telomerization which is defined as a organic reaction involving addition of fragments of one molecule such as an alcohol, acetal, or chloroform, to the ends of a polymerizing olefin system, for example the reaction of carbon tetrachloride with styrene in the presence of acetyl peroxide to form the telomers Cl{CH(C₆H₅)cCH₂}ₙCCl₃.

Examples of such organic molecules, telomers, and polymers are: cellulose, polystyrene, crown ethers, polydimethylsiloxanes, glycol polymers, fatty acids, fatty esters, borate esters, carbohydrates, optically active enantiomers, polyolefins grafted with allylglycidylether, enzymes, polyethyleneimines, vitamins, especially vitamin E, poly(acrylic acid), poly(acrylic acid) derivatives, non-ionic surfactants, copolymers of allyglycidylether with monomers, organic amines,

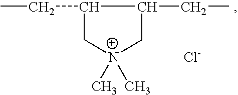

The solid substrates include any of the solid materials exhibiting a reactive surface that can react with the silanes of this invention, that is, a reaction wherein stable covalent bonding will occur.

In another embodiment of this invention, high molecular weight molecules and polymers, that contain an aliphatic C—H unit, may be immobilized onto polymeric, particulate materials such as polyethylene and polystyrene, that contain an aliphatic C—H unit by utilizing the free radical catalyzed addition reaction of the aliphatic C—H group to a diene such as butadiene or octadiene, as the crosslinker, to achieve a covalent bond.

Any free radical generating technique including peroxides, radiation, mechanical energy, and the like may be used to catalyze the addition of the aliphatic C—H to the olefinic functional group.

Conditions for reacting the silanes such as chlorosilanes, alkoxysilanes, acetoxysilanes, and the like, onto the solid substrate are well-known techniques and such preparations do not need to be set forth herein.

A broad range of final product compositions and structures may be accomplished by stoichiometric control of the addition of the materials and further reaction of the immobilized materials to additional monomers or polymers, including the same macrornonomers or polymers or a different macromonomer or polymer containing an aliphatic (C—H group) via use of free radical catalyzed reaction of dienes such as octadiene. For purposes of this invention, the value of each of n and m is from 2 to 2,000. “Higher molecular weight” for this invention means a value of about 15,000 to about 50,000 (n=about 21 to 2000) and “low molecular weight” for this invention means a value of from 800 to less than 15,000 (n=about 2 to 20).

A wide variety of applications exist for the materials disclosed herein including chromatographic, both liquid, and gas/liquid systems, catalysts for both organic processes and biological processes, various industrial uses, dairy applications, photographic applications, optical applications, brewery processes, leather treating, textile applications, pharmaceutical applications, catalytic materials stabilization, metal capture through chelation, purification and separation applications, personal care products, household care products, defoamers and antifoams, polymer modifiers, and the like.

### EXAMPLES

Vinylsilane bonded silicas were prepared by reaction of vinyltrimethoxysilane or vinyltrichlorosilane with various silicas using known procedures.

### Example 1

About 10 g of vinyl silane bonded silica was suspended in 200 ml of benzene, and there was added to the suspended bonded silica, 7 g of 100,000 molecular weight of polystyrene dissolved in 50 ml of benzene. To this mixture was added 0.8 g of benzoyl peroxide and then it was heated for about 12 hours at 80° C. while stirring.

The modified bonded silica was isolated by using washing and drying techniques to give polystyrene bonded to the silica.

The silica was packed in an HPLC column and a run was made on the HPLC and it showed polystyrene bonded to silica.

### Example 2

To a mixture of 5 g of vinylsilane bonded to silica as produced in Example 1, there was added 200 ml of benzene, and 2.2 g of 18-crown-6-ether and 0.2 g of benzoyl peroxide. The mixture was reacted t 80° C. for about 12 hours.

Upon isolation and drying of the silica, 18-crown-6 ether bonded to silica was obtained. HPLC showed the 18-crown-6 ether bonded to silica.

### Example 3

To a mixture of 5 g of vinylsilane bonded silica in 200 ml of benzene, there was added 2 g of (+)-α-topcopherol and 0.05 g of benzoyl peroxide. The mixture was reacted at 65° C. for 12 hours.

Upon isolation and drying of the resulting silica, vitamin E immobilized silica was obtained.

### Example 4

To a mixture of 10 g of polystyrene and 2 g of allylglycidylether in 150 ml of benzene, there was added 0.02 g of benzoyl peroxide. The mixture was reacted at 65° C. for 12 hours. Into the reacted mixture there was added 10 g of vinylsilane bonded silica and 0.015 g of benzoyl peroxide. The mixture was reacted at 70° C. for 12 hours.

Upon isolation and drying of the resulting silica, immobilized allylglycidylether grafted polystyrene on silica was obtained.

### Example 5

A mixture of 5 g of immobilized allylglycidylether grafted polystyrene on silica from example 4, and 4 g of papain in buffered solution was stirred at 40 to 50° C. for 12 hours.

Upon isolation, washing with deionized water, and drying, papain immobilized silica was obtained. It is believed that the reaction involved the epoxy groups reacting with the amino groups of the papain.

### Example 6

To a mixture of 150 ml of poly(methyl acrylamide) in water (10%) and 5 g of vinylsilane bonded silica, there was added 0.02 g of benzoyl peroxide. The mixture was reacted at 70° C. for 12 hours.

Upon isolation and drying of the resulting silica, poly(methylacrylamide) immobilized silica was obtained.

### Example 7

Following the procedure of example 1, the following molecules and polymers were immobilized on silica via covalent bonding.
polydimethylsiloxane fluid
poly(ethylene glycol)
fatty acid (C₆)
fatty ester (C₂₄)
dextrin
papain*
allylglycidylether grafted polyethylene
polyethyleneimine*
poly(diallyldimethylammonium chloride)
poly(vinylmethyl)ketone
poly(acrylamide)
poly{bis-(2-chloroethyl)ether-alt-1,3-bis-(3-(dimethylamino)propyl)urea}, quaternized
D-Mannose** and
Heparin**,
   wherein * denotes that water was used as a solvent and ** denotes that buffered water was used as the solvent.

### Example 8

A mixture of 5 g of immobilized allylglycidylether grafted polystyrene on silica from example 4 and 2 g of ampicillin were reacted in 100 ml of benzene at 50° C. for 10 hours.

Upon isolation, washing with methanol and drying, ampicillin immobilized silica via the reaction of epoxy groups and amino groups of ampicillin, was obtained. An HPLC of the silica showed chirality.

### Example 9

A mixture of 10 g of crosslinked polystyrene beads, 3 g of allylglycidylether, and 0.02 g of benzoyl peroxide in 100 ml of benzene was reacted at 65° C. for 12 hours.

Upon isolation and drying of polystyrene beads, allylglycidylether grafted polystyrene beads were obtained.

The grafted polystyrene beads were found to react with polyethyleneimine in DFM solution to yield polyethyleneimine immobilized polystyrene beads via the reaction of epoxy groups with amino groups of the polyethyleneimine.

### Example 10

A mixture of 5 g of poly(methylacrylate) immobilized silica of Example 6 and 3 g of polyethyleneimine in 50 ml of DMF was reacted at 40 to 50° C. for 10 hours.

Upon isolation, washing with methanol, and drying, polyethyleneimine immobilized silica, via reaction of ester groups and amino groups, was obtained.

### Example 11

The immobilized products of example 2, example 5, example 6, and example 10 were selected and tested for chelating capability with copper sulfate, and found to be chelating agents.

### Example 12

A mixture of immobilized allylglycidylether-grafted polystyrene on silica from example 4, and 2 g of 2-(aminomethyl-15-crown-5 was reacted in 100 ml of benzene at 50° C. for 10 hours.

Upon isolation, washing with methanol and drying, the amino-crown ether-immobilized silica via the reaction of epoxy groups and amino groups of the crown ether is obtained

1. A method of covalently bonding a material selected from the group consisting of
i organic molecules,
ii telomers, and
iii polymers,
on solid substrates, the process comprising:
(I) contacting the solid substrate with an alkenyl containing silane containing organofunctional moieties and allowing the organofunctional moieties to react with the solid substrate to provide an alkenyl functional group bound to the solid substrate;
(II) contacting the product of (I) with a material selected from the group consisting of
i organic molecules,
ii telomers, and
iii polymers,
to covalently bond the material to the solid substrate.

2. A method of covalently bonding a material as claimed in claim 1 where, in addition, there is a source of free radicals present.

3. A method as claimed in claim 2 wherein the source for the free radical generation is a peroxide.

4. The method as claimed in claim 2 wherein the source for the free radical generation is radiation.

5. The method as claimed in claim 2 wherein the source for the free radicals is mechanical energy.

6. A composition of matter prepared by the process of claim 1.

7. A composition of matter prepared by the process of claim 2.

8. A method as claimed in claim 1 wherein (i), (ii), and (iii) are selected from the group consisting of:
(i) cellulose, (ii) polystyrene, (iii) crown ethers, (iv) polydimethylsiloxanes, (v) glycol polymers, (vi) fatty acids, (vii) fatty esters, (viii) borate esters, (ix) carbohydrates, (x) optically active enantiomers, (xi) polyolefins grafted with allylglycidylether, (xii) enzymes, (xiii) polyethyleneimines, (xiv) vitamin E, (xv) poly(acrylic acid) (xvi) poly(acrylic acid) derivatives, (xvii) non-ionic surfactants, (xviii) copolymers of allyglycidylether with monomers, (xix) organic amines, 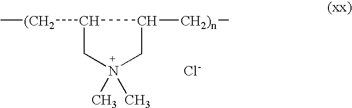
wherein the value of n ranges from 2 to 100,000; 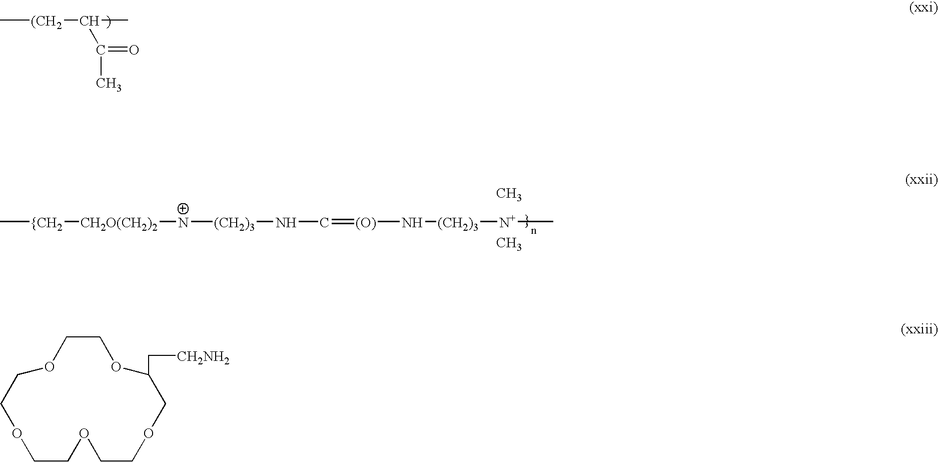
(xxiv) alcohols, and (xxv) polyols.

9. A method as claimed in claim 2 wherein (i), (ii), and (iii) are selected from the group consisting of:
(i) cellulose, (ii) polystyrene, (iii) crown ethers, (iv) polydimethylsiloxanes, (v) glycol polymers, (vi) fatty acids, (vii) fatty esters, (viii) borate esters, (ix) carbohydrates, (x) optically active enantiomers, (xi) polyolefins grafted with allylglycidylether, (xii) enzymes, (xiii) polyethyleneimines, (xiv) vitamin E, (xv) poly(acrylic acid) (xvi) poly(acrylic acid) derivatives, (xvii) non-ionic surfactants, (xviii) copolymers of allyglycidylether with monomers, (xix) organic amines, 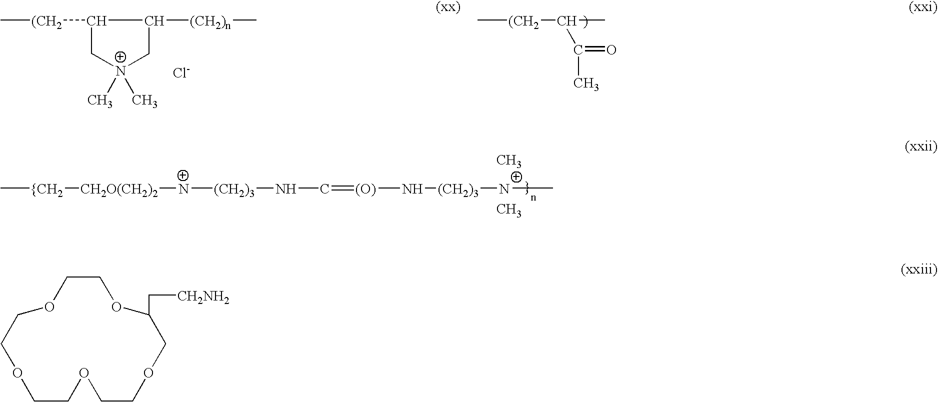
(xxiv) alcohols, and (xxv) polyols.

10. A method as claimed in claim 8 wherein the polystyrene is polystyrene grafted with allylglycidylether.

11. A method as claimed in claim 9 wherein the polystyrene is polystyrene grafted with allylglycidylether.

12. A method as claimed in claim 8 wherein the crown ethers are selected from the group consisting of:

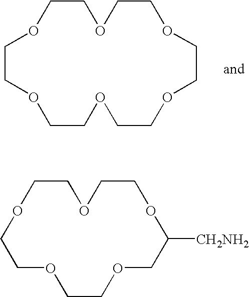

13. A method as claimed in claim 9 wherein the crown ethers are selected from the group consisting of:

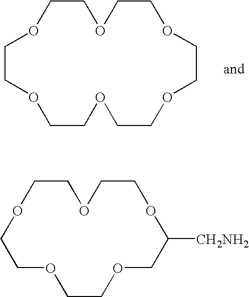

14. A method as claimed in claim 8 wherein the enzyme is papain.

15. A method as claimed in claim 9 wherein the enzyme is papain

16. A method in which materials selected from the groups consisting of
(i) organic molecules,
(ii) telomers, and
(iii) polymers
are (I) covalently reacted, in the presence of a free radical initiator, with an alkenyl group on a silane, wherein the silane also contains organofunctional moieties, and then
(II) reacting the product of (I) with a solid substrate using the organofunctional moieties to bind the solid substrate to the product.

17. A composition of matter prepared by the process of claim 16.

18. A personal care product that is a formulated product that includes a composition of claim 6.

19. A personal care product that is a formulated product that includes a composition of claim 7.

20. A personal care product that is a formulated product that includes a composition of claim 17.

21. A defoamer composition that is a formulated product that includes a composition of claim 6.

22. A defoamer composition that is a formulated product that includes a composition of claim 7.

23. A defoamer composition that is a formulated product that includes a composition of claim 17.

24. An antifoam product that is a formulated product that includes a composition of claim 6.

25 An antifoam product that is a formulated product that includes a composition of claim 7.

26. An antifoam product that is a formulated product that includes a composition of claim 17.

27. A method of chelating metals from aqueous solutions, the method comprising, moving the aqueous solution into contact with a composition of claim 6.

28. A method of chelating metals from aqueous solutions, the method comprising, moving the aqueous solution into contact with a composition of claim 7.

29. A method of chelating metals from aqueous solutions, the method comprising, moving the aqueous solution into contact with a composition of claim 17.

30. A catalyzed process, wherein the composition of claim 6 is used as the catalyst.

31. A catalyzed process as claimed in claim 30 that is a organic process.

32. A catalyzed process as claimed in claim 30 that is a biological process.

33. A catalyzed process, wherein the composition of claim 7 is used as the catalyst.

34. A catalyzed process as claimed in claim 33 that is a organic process.

35. A catalyzed process as claimed in claim 33 that is a biological process.

36. A catalyzed process wherein the composition of claim 17 is used as the catalyst.

37. A catalyzed process as claimed in claim 36 that is a organic process.

38. A catalyzed process as claimed in claim 36 that is a biological process.

39. A liquid chromatographic process wherein a composition of claim 6 is used as the chromatographic support.

40. A liquid chromatographic process wherein a composition of claim 7 is used as the chromatographic support.

41. A liquid chromatographic process wherein a composition of claim 17 is used as the chromatographic support.
